(19) United States
(12) Patent Application Publication
(10) Pub. No.: US 2007/0167810 A1
(43) Pub. Date: Jul. 19, 2007

(54) ULTRASONIC PROBE PROTECTION BRACKET AND ULTRASONIC PROBE

(76) Inventor: Hiroki Ito, Tokyo (JP)

Correspondence Address: Patrick W. Rasche;Armstrong Teasdale LLP, Suite 2600, One Metropolitan Square, St. Louis, MO, 63102, US

(21) Appl. No.: 11/612,635

(22) Filed: Dec. 19, 2006

(30) Foreign Application Priority Data
Dec. 28, 2005 (JP) 2005-378010

(51) Int. Cl. A61B 8/14

(52) U.S. Cl. 600/459

(57) ABSTRACT

An ultrasonic probe protection bracket for performing disinfection of a probe without damage to a lens at a probe end is provided. An ultrasonic probe protection bracket for protecting a lens at an end of an ultrasonic probe. In a state where the bracket is attached to the end portion of the probe, the probe end portion is accommodated inside the bracket thereby the lens is protected, and a gap exists between the probe and the bracket. The bracket has an engagement member on an inner wall of its end portion to be engaged with an engagement member of the probe, and an accommodation member to protect the probe end portion in its rear end portion. In a state where the bracket is attached to the probe, the accommodation member rear end of the bracket is longer than the position of the end of the lens of the probe, and the end of the lens is positioned inside the accommodation member of the bracket.

### BACKGROUND OF THE INVENTION

The present invention relates to a protection bracket for an ultrasonic diagnostic apparatus probe (hereinbelow, referred to as an ultrasonic probe), and more particularly, to a bracket used to protect an acoustic device array (hereinbelow, may also be referred to as a lens) provided at an end of the probe in a disinfection/cleaning process or the like of the probe, and an ultrasonic probe in which the bracket is attached to its end.

An ultrasonic diagnostic apparatus is widely used in implementation of ultrasonic image processing and measurement of human body by using a probe placed inside or outside the body to be subjected to inspection. The probe originates energy pulses in the body, and they are transmitted/received with an acoustic device array to receive a return pulse of the energy reflected from the internal structure of the body. The pulse or beam of the return energy is electrically transferred to the ultrasonic diagnostic apparatus.

As a probe, a diagnosis probe for execution of general ultrasonic diagnosis, a paracentesis probe for execution of paracentesis under the guidance by the ultrasonic diagnostic apparatus, a surgical probe, a scan head of which is placed inside the body during surgery such as vascular or transplant surgery, and the like, are known. To use these probes for different patients, it is necessary to disinfect (sterilize, pasteurize) the probes.

In the probe disinfection process, in some cases, the lens at the probe end is damaged, or broken, and the impact of the breakage destroys the acoustic device. Especially, when the length of the probe for diagnosis is long as in the case of an endovaginal probe, there is a probability that the lens is broken due to handling upon disinfection.

### SUMMARY OF THE INVENTION

Therefore, an object of the present invention is to provide a probe protection bracket which is mainly used upon disinfection of the probe for disinfection without damage to the lens at the probe end.

Further, the present invention also has an object to provide a probe protection bracket which is attached when there is a probability that the lens provided at the end of the probe is damaged upon conveyance or the like of the probe.

Further, the invention also has an object to provide an ultrasonic probe which can be subjected to disinfection without damaging the lens.

The ultrasonic probe protection bracket of the present invention is an ultrasonic probe protection bracket to protect a lens at an end of an ultrasonic probe, wherein, in a state where the bracket is attached to a probe end portion, the probe end portion is placed inside the bracket thereby the lens is protected, and a gap exists between the probe and the bracket.

It is preferable that the bracket has an engagement member that is engaged with an engagement member of the probe, on an inner wall of its end portion, and an accommodation member that protects the probe end portion, at its rear end portion, and wherein, in a state where the bracket is attached to the probe, the rear end of the bracket accommodation member is longer than the position of a lens end of the probe, and the lens is positioned inside the accommodation member at the end of the bracket.

Further, in the ultrasonic probe of the present invention, the ultrasonic protection bracket is attached to the end.

Since the bracket of the present invention protects the lens at the end of the probe upon disinfection or the like of the probe and does not prevent disinfectant medium from contact with the probe, the disinfection of the probe can be efficiently performed. Further, the bracket of the present invention is attached to the probe when there is a probability that the lens provided at the end of the probe is damaged upon conveyance or the like of the probe, and the probe end can be protected. Further, in the ultrasonic probe in which the bracket is attached to its end, the lens at the probe end is protected, and further, as disinfectant medium becomes into contact with the probe main body, disinfection of the probe can be efficiently performed.

Further objects and advantages of the present invention will be apparent from the following description of the preferred embodiments of the invention as illustrated in the accompanying drawings.

### BRIEF DESCRIPTION OF THE DRAWINGS

FIG. 1 is a perspective view showing a state where the probe and the bracket showing the working example of the present invention are separated.

FIG. 2 is a cross-sectional view showing a state where the probe and the bracket as the working example are attached.

FIG. 3 is a cross-sectional view showing a state where the probe and the bracket as the other working example are attached.

FIG. 4 is a cross-sectional view upon disinfection of the ultrasonic probe of the present invention with disinfectant liquid.

### DETAILED DESCRIPTION OF THE INVENTION

An ultrasonic probe has a convex type lens having a curved probe surface or a linear type lens having a flat probe surface. The bracket of the present invention especially produces a high degree of effectiveness when it is adapted to the convex type probe where the lens surface is vulnerable. However, the bracket is also effective for the linear surface type lens. Since the both types of lenses are not particularly distinguished from each other, the lens will be simply represented as a lens in the following description of the working example.

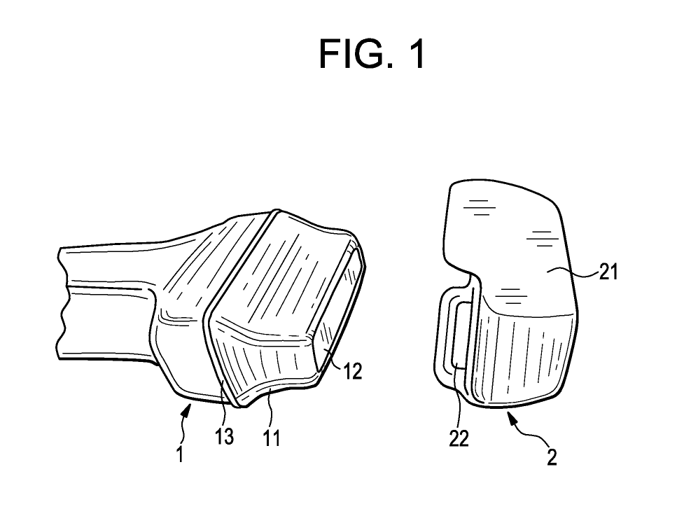

FIG. 1 shows an ultrasonic probe 1 and a bracket 2 according to a working example of the present invention. The ultrasonic probe 1 comprises a probe main body 11 and a lens 12 attached to its end. The lens 12 is projected from a case end surface of the probe main body 11, to be in contact with a subject more efficiently. However, as the surface of the lens is projected, the lens surface is at risk of damage due to contact or collision with another material. Particularly, upon disinfection or cleaning of the probe, there is a high probability that the lens is damaged due to contact or collision with a cleaning tool. Further, in the case of an endovaginal probe having a long length, there is a high probability that the end of the probe main body 11 collides with another object.

An engagement member 13 is provided on a side wall part of the probe main body 11. The engagement member 13 shown in FIG. 1 is formed with a convex rail surrounding an outer wall of the probe main body. The engagement member 13 having the convex rail is engaged with the bracket 2 to be described later.

The bracket 2, which protects the lens 12 of the ultrasonic probe 1, comprises an accommodation member 21 to protect the lens 12 and an engagement member 22 having a concave groove shape to be engaged with the convex rail engagement member 13 of the ultrasonic probe 1.

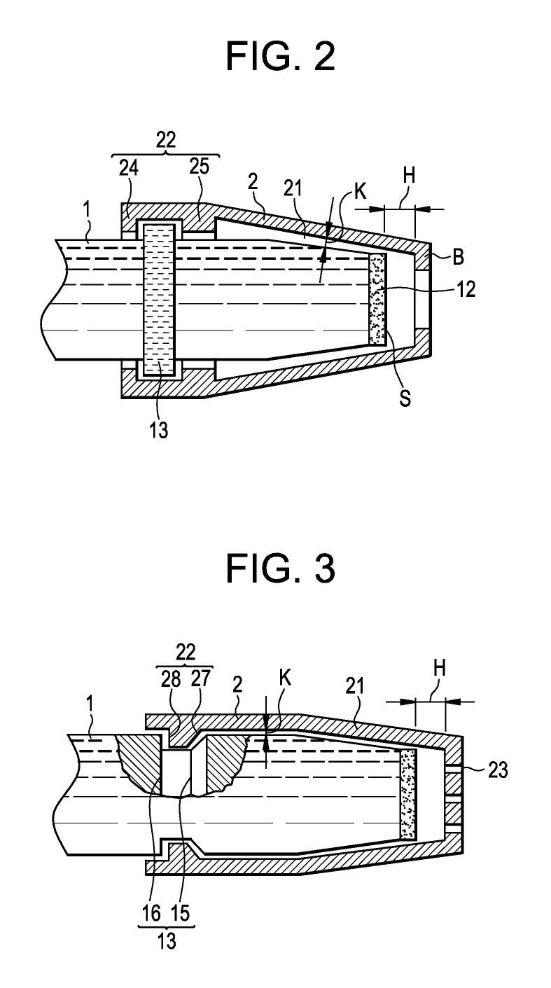

FIG. 2 is a cross-sectional view showing a state where the bracket 2 is attached to the ultrasonic probe 1. As shown in the figure, an accommodation member (hereinbelow, referred to as an accommodation member) 21 to accommodate the lens of the bracket 2 protects the lens 12 of the ultrasonic probe 1. As shown in FIG. 2, an end S of the lens 12 and a rear end B of the accommodation member 21 of the bracket 2 are formed such that the rear end B of the accommodation member 21 of the bracket 2 is longer by a length H. In this manner, the rear end B of the accommodation member 21 of the bracket 2 is projected, thereby the rear end B of the accommodation member 21 is outwardly longer than the end surface S of the lens 12 by H, such that, when the surface of the lens 12 is to become into contact with a wall surface of a disinfection/cleaning tool, the accommodation member rear end B of the bracket 2 becomes into contact with the tool and protects the lens 12.

The accommodation member 21 may have an arbitrary shape as long as it can cover the portion of the lens 12 and protect the lens 12. For example, as shown in FIG. 2, it has a hood type shape, or as shown in FIG. 3, a wall surface with one or plural through holes 23 for immersion in disinfectant/cleaning medium. Note that the hole 23 has an arbitrary size as long as it allows incomings and outgoings of the disinfectant medium. Further, it may be arranged such that the through holes are provided in wall surfaces of a case type accommodation member to improve circulation of the disinfectant liquid.

Further, to fully bring the disinfectant liquid or the like into contact with the ultrasonic probe 1, a gap K in which at least liquid enters is provided between the ultrasonic probe 1 and the bracket. In this manner, as the gap K is provided, the liquid such as disinfectant liquid is fully brought into contact with the surface of the ultrasonic probe 1. Further, when a collision occurs with the bracket 2, the gap K works as a cushion to mitigate the impact transmitted to the ultrasonic probe 1.

The engagement member 22 of the bracket 2 shown in FIG. 2 is formed to have a concave groove shape, and as shown in the figure, the height of an outside groove 24 is lower than an inside groove 25. The heights are set such that when the concave-groove shaped engagement member 22 is engaged with the engagement member (in FIG. 2, a convex rail engagement member) 13 of the ultrasonic probe 1, the outside groove 24 climbs over the height of the convex rail engagement member 13 against the elastic force of the bracket 2 but the inside groove 25 cannot climb over the convex rail engagement member 13. That is, when the bracket 2 is attached to the ultrasonic probe 1, the outside groove 24 of the bracket 2 is pressed against the convex rail engagement member 13 of the ultrasonic probe 1 and further pushed in, thereby the outside groove 24 climbs over the convex rail engagement member 13 but the inside groove 25 cannot climb over the convex rail engagement member 13, and the bracket 2 is engaged with the ultrasonic probe 1. Note that when the bracket is removed from the ultrasonic probe 1, the bracket 2 is pulled out so as to be moved away from the ultrasonic probe 1, thereby the bracket can be removed. As shown in the figure, the engagement members 13 and 22 are provided as a pair in upper and lower positions, and the bracket 2 is engaged such that the bracket holds the ultrasonic probe 1.

As described above, in a state where the ultrasonic probe 1 and the bracket 2 are engaged with engagement between the convex rail engagement member 13 and the concave groove engagement member 22, the surface of the lens 12 is protected with the accommodation member 21, and there is no probability that the surface of the lens 12 is brought into direct contact with an outer wall or the like.

FIG. 3 shows another working example of the engagement members 13 and 22. In FIG. 3, the engagement member 13 on the side of the ultrasonic probe 1 has a concave groove shape, and it is sloped in a direction where the width of a groove 15 of the lens 12 side becomes wider, and a groove 16 on the opposite side to the lens 12 is vertical to the probe main body 11. On the other hand, the engagement member 22 on the bracket side has a convex rail shape. An outside surface 27 of the convex rail on the side of the accommodation member 21 is sloped such that the width of its end becomes narrower, and the opposite side 28 to the accommodation member 21 is vertical to the bracket. As the engagement members are formed in this manner, the ultrasonic probe 1 and the bracket 2 can be easily attached/detached.

To disinfect/clean the ultrasonic probe 1, disinfectant medium such as disinfectant liquid or cleaning liquid is used. To completely disinfect/clean the probe 1, it is necessary to fully bring the disinfectant medium into contact with necessary portions of the probe 1 in a state where the bracket 2 is attached. Accordingly, when the bracket 2 is attached to the ultrasonic probe 1, if the bracket 2 prevents contact between the disinfectant liquid/cleaning liquid and the ultrasonic probe 1, disinfection/cleaning is insufficient. As described above, the bracket 2 of the present invention has a gap for entrance of the disinfectant medium between the ultrasonic probe 1 and the bracket 2.

The width of the gap is sufficient as long as the disinfectant medium can be fully brought into contact with the ultrasonic probe 1. That is, the size of the accommodation member 21 of the bracket 2 is slightly larger than the size of the ultrasonic probe 1, and when it is attached to the ultrasonic probe 1, the gap K where at least the disinfectant medium enters exists around the member and between the member and the probe main body 11 and has a length H to protect the lens 12. In the working embodiment shown in FIG. 2, the disinfectant medium can freely enter the hood type accommodation member 21. In the accommodation member 21 having the through holes 23 shown in FIG. 3, the disinfectant medium can be fully brought into contact with the probe main body 11 via the through holes 23.

Further, when it is necessary to disinfect the engagement members 13 and 22 as concave rail engagement member and convex groove engagement member, the width of the convex engagement member is narrower than the groove width of the concave groove engagement member. Further, the height of the convex engagement member is lower than the height of the concave groove such that the members are loosely engaged with each other and the disinfectant medium can be fully brought into contact with the engagement members 13 and 22.

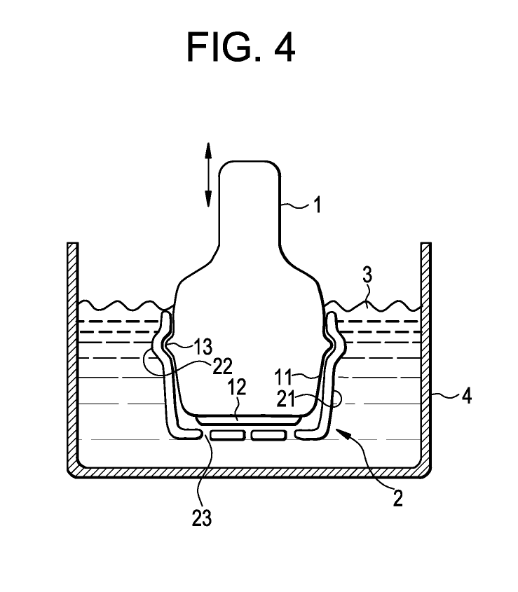

The disinfection of the ultrasonic probe 1 will be described with reference to FIG. 4.

As shown in FIG. 4, the bracket 2 is attached to the ultrasonic probe 1 by engagement between the engagement members 13 and 22, and immersed in a disinfection container 4 filled with disinfectant liquid 3 as a disinfectant medium or cleaning liquid. Upon disinfection, the disinfectant liquid 3 enters a gap between the bracket 2 and the probe main body 11 of the ultrasonic probe 1, further, enters the space between the engagement members 13 and 22, and disinfects the ultrasonic probe 1. At this time, as the surface of the lens 12 exposed at the end of the ultrasonic probe 1 is protected with the accommodation member 21, the surface of the lens 12 is not brought into contact with the wall surface of the disinfection container 4, and there is no probability of damage to the surface of the lens 12, and further, disinfection can be fully performed. Further, in a cleaning process following the disinfection process, the cleaning medium circulates around the ultrasonic probe 1, and the ultrasonic probe 1 can be cleaned without being damaged.

In the above description, the disinfection/cleaning process in which the bracket 2 is attached to the ultrasonic probe 1 and liquid is used as a disinfectant medium has been mainly described, however, in the case of gas sterilization using gas as a disinfectant medium on the ultrasonic probe 1, a similar effect to that upon the liquid disinfection can be obtained. Note that when the bracket 2 is employed only for gas sterilization, the width of the gap between the ultrasonic probe 1 and the bracket 2 may be set such that the gas as the disinfectant medium pervades the gap. Accordingly, the gap K can be narrowed.

FIG. 1 shows the working example where the present invention is applied to a linear type probe surface, however, the invention can also be applicable to a convex type probe surface, and further, can be adopted for protection of probe surface regardless of probe surface shape.

Further, the bracket 2 of the present invention can be applied regardless of type of probe such as a general diagnosis probe, a paracentesis probe for execution of paracentesis, a surgical probe and the like. Especially, when the bracket 2 of the present invention is applied to an endovaginal probe having a long length, as the entire length of the probe main body including the probe end can be covered with the bracket, the probe breakage can be effectively prevented.

The subject matter of the bracket 2 of the present invention is to protect the lens exposed at the end of the ultrasonic probe 1 and prevent damage to the lens by application of impact to the lens. However, the bracket also effectively prevents breakage of the lens 12 of the ultrasonic probe 1 by application of impact to the lens. For example, when the ultrasonic probe 1 is conveyed in a state where the bracket 2 is attached, it can be conveyed while the lens is protected from impact or the like upon conveyance.

Many widely different embodiments of the invention may be configured without departing from the spirit and the scope of the present invention. It should be understood that the present invention is not limited to the specific embodiments described in the specification, except as defined in the appended claims.

1. An ultrasonic probe protection bracket to protect a lens at an end of an ultrasonic probe, wherein, in a state where the bracket is attached to a probe end portion, the probe end portion is placed inside the bracket thereby the lens is protected, and a gap exists between the probe and the bracket.

2. The ultrasonic probe protection bracket according to claim 1, wherein the bracket has an engagement member that is engaged with an engagement member of the probe, on an inner wall of its end portion, and an accommodation member that protects the probe end portion, at its rear end portion, and wherein, in a state where the bracket is attached to the probe, the rear end of the bracket accommodation member is longer than the position of a lens end of the probe, and the lens is positioned inside the accommodation member at the end of the bracket.

3. The ultrasonic probe protection bracket according to claim 1, wherein the rear end of the accommodation member of the bracket is opened.

4. The ultrasonic probe protection bracket according to claim 1, wherein the rear end of the accommodation member of the bracket is formed with a wall surface having one or plural through holes.

5. The ultrasonic probe protection bracket according to claim 1, wherein at least the accommodation member of the bracket is formed with a wall surface having plural through holes.

6. The ultrasonic probe protection bracket according to claim 1, wherein opposing inner walls of the end portion of the bracket have an engagement concave groove that is engaged with an engagement projection provided on a probe main body.

7. The ultrasonic probe protection bracket according to claim 6, wherein the engagement groove is provided on the opposing inner walls of the bracket, and wherein the distance between opposing groove crests of the engagement groove is longer than the width of the probe main body, and shorter than the distance between crests of a pair of engagement projections provided on the probe.

8. The ultrasonic probe protection bracket according to claim 1, wherein opposing inner walls of the end portion of the bracket have an engagement projection that is engaged with an engagement concave groove provided on a probe main body.

9. An ultrasonic probe comprising:
an end portion; and
an ultrasonic probe protection bracket coupled to said end portion, wherein, in a state where said probe protection bracket is attached to said probe end portion, said probe end portion is placed inside said probe protection bracket such that a lens is protected and such that a gap is defined between said probe and said probe protection bracket.

10. An ultrasonic probe protection bracket for an ultrasonic probe, said ultrasonic probe protection bracket comprising:
an accommodation member; and
an engagement member formed with said accommodation member, said engagement member is configured to couple said accommodation member to an ultrasonic probe such that said accommodation member substantially circumscribes the ultrasonic probe and such that a gap is defined between said accommodation member and an ultrasonic probe lens.

11. An ultrasonic probe protection bracket in accordance with claim 10, wherein said engagement member comprises:
an outside groove having a first diameter; and
an inside groove having a second diameter that is less than the first diameter, said inside and outside grooves are configured to secure said ultrasonic probe protection bracket to the ultrasonic probe.

12. An ultrasonic probe protection bracket in accordance with claim 11, wherein said ultrasonic probe engagement member comprises a convex rail engagement member having a third diameter that is greater than the inside and outside groove diameters such that said inside and outside grooves are configured to secure said ultrasonic probe protection bracket to the ultrasonic probe.

13. An ultrasonic probe protection bracket in accordance with claim 10, wherein said gap is defined between an end wall of said accommodation member and an ultrasonic probe lens.

14. An ultrasonic probe protection bracket in accordance with claim 12, wherein said gap extends between said end wall and said convex rail engagement member.

15. An ultrasonic probe protection bracket in accordance with claim 13, further comprising a single opening extending through said end wall.

16. An ultrasonic probe protection bracket in accordance with claim 13, further comprising a plurality of openings extending through said end wall.

17. An ultrasonic probe protection bracket in accordance with claim 10, wherein said accommodation member has a cross-sectional profile that is substantially similar to a cross-sectional profile of at least a portion of the ultrasonic probe.

18. An ultrasonic probe protection bracket in accordance with claim 10, wherein said accommodation member comprises:
an end wall;
a pair of opposing inner walls extending from and coupled to said end wall; and
an engagement concave groove that is formed in said pair of opposing inner walls, said engagement concave groove is configured to engage an engagement projection provided on a probe main body.

19. An ultrasonic probe protection bracket in accordance with claim 10, wherein said end wall is fabricated from a flexible material to enable said end wall to flex when contacted with an object to facilitate protecting an ultrasonic probe lens.

20. An ultrasonic probe protection bracket in accordance with claim 10, wherein said accommodation probe has an inner diameter that is larger than an outer diameter of a portion of the ultrasonic probe that is inserted within said accommodation member.
